(19) United States
(12) Patent Application Publication
(10) Pub. No.: US 2007/0238672 A1
(43) Pub. Date: Oct. 11, 2007

(54) CO-ADMINISTRATION OF ADENOSINE A1 RECEPTOR ANTAGONISTS AND ANTICONVULSANTS

(76) Inventors: Howard Dittrich, San Diego, CA (US); Eckard Weber, San Diego, CA (US); Brian Farmer, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/696,623

(22) Filed: Apr. 4, 2007

Related U.S. Application Data
(60) Provisional application No. 60/789,843, filed on Apr. 6, 2006.

(51) Int. Cl. A61K 31/7008; A61K 31/5513; A61K 31/55; A61K 31/522; A61K 31/515; A61K 31/195; A61K 31/19

(52) U.S. Cl. 514/23; 514/221; 514/263.32; 514/557; 514/389; 514/217; 514/270; 514/561

(57) ABSTRACT

Disclosed herein are pharmaceutical compositions that include an AA₁RA, or a salt, ester, amide, metabolite, or prodrug thereof, and an anticonvulsant agent. Also disclosed are methods of treating patients suffering from congestive heart failure, methods of improving renal function, and methods of restoring renal function comprising the step of administering a therapeutically effective amount of an AA₁RA or a salt, ester, amide, metabolite, or prodrug thereof, in combination with an anticonvulsant agent.

### RELATED APPLICATIONS

The present application claims priority to U.S. Provisional Application Ser. No. 60/789,843, filed on Apr. 6, 2006, by Dittrich et al., and entitled “CO-ADMINISTRATION OF ADENOSINE A1 RECEPTOR ANTAGONISTS AND ANTICONVULSANTS;” the entire disclosure of which is herein incorporated by reference in its entirety.

### FIELD OF THE INVENTION

The present invention relates to compositions comprising a combination of an adenosine A₁ receptor antagonist and an anticonvulsant and methods of treating patients with said compositions.

### SUMMARY OF THE INVENTION

Provided herein are pharmaceutical compositions comprising an effective prophylactic amount of anticonvulsant and an adenosine A₁ receptor antagonist (AA₁RA). Also provided herein are improved methods of providing AA₁RA therapy to patients, by administering an AA₁RA and an anticonvulsant to the patient.

In some embodiments, the compositions disclosed herein can include any one of the following anticonvulsants, or any combination thereof: diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, or zonisamide. Preferably, the anticonvulsant is a benzodiazepine, such as diazepam or lorazepam.

In some embodiments, the compositions disclosed herein can include an AA₁RA that is a xanthine-derivative compound of Formula I or a pharmaceutically acceptable salt thereof, 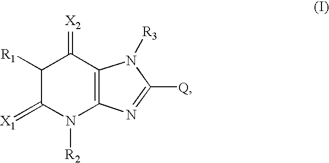
wherein each of X₁ and X₂ independently represents oxygen or sulfur; Q represents 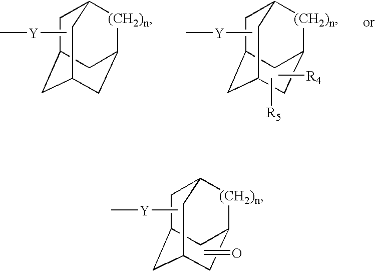
and where Y represents a single bond or alkylene having 1 to 4 carbon atoms, n represents 0 or 1, then each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or R₄ and R₅ are the same or different and each represent hydrogen or hydroxy, and when both R₄ and R₅ are hydrogen, at least one of R₁ and R₂ is hydroxy-substituted or oxo-substituted lower alkyl, provided that when Q is 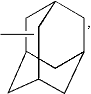
then R₁, R₂ and R₃ are not simultaneously methyl.

In some embodiments, both of R₁ and R₂ are lower alkyl and R₃ can be hydrogen; and both of X₁ and X₂ can be oxygen.

In some embodiments, each of R₁, R₂ and R₃ can independently represent hydrogen or lower alkyl. In further embodiments, Q can be 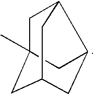
In other further embodiments, Q can be

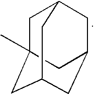

In some embodiments, each of R₁ and R₂ can independently represent allyl or propargyl and R₃ can represent hydrogen or lower alkyl. In further embodiments, X₁ and X₂ can both be oxygen and n can be 0.

In some embodiments, R₁ can be a hydroxy-substituted, oxo-substituted or unsubstituted propyl; R₂ can be a hydroxy-substituted or unsubstituted propyl; and Y can be a single bond.

In some embodiments, R₁ can be propyl, 2-hydroxypropyl, 2-oxopropyl or 3-oxopropyl; R₂ is propyl, 2-hydroxypropyl or 3-hydroxypropyl.

In some embodiments Q can be 9-hydroxy, 9-oxo or 6-hydroxy substituted 3-tricyclo[3.3.1.0^{3,7}]nonyl, or 3-hydroxy-1tricyclo[3.3.1.1^{3,7}]decyl.

In some embodiments, the AA₁RA can be selected from one of the following: 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine, 8-(cis-9-hydroxy-3-tricyclo[3.3.1.0^{3,7}]nonyl)-1,3-dipropylxanthine, 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine, or a pharmaceutically acceptable salt thereof. Preferably, the AA₁RA is KW-3902.

In other embodiments, the AA₁RA can be a xanthine epoxide-derivative compound of Formula II or Formula III, or a pharmaceutically acceptable salt thereof,

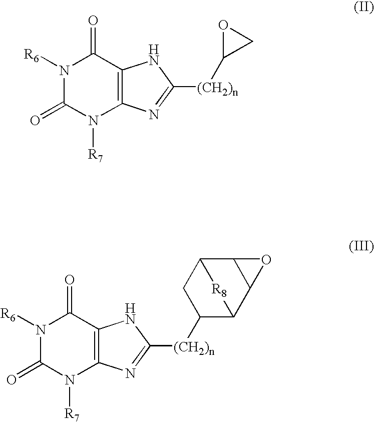

wherein R₆ and R₇ are the same or different, and can be hydrogen or an alkyl group of 1-4 carbons, R₈ is either oxygen or (CH₂)₁₋₄, and n=0-4.

For example, in some embodiments, the xanthine epoxide-derivative compound can be

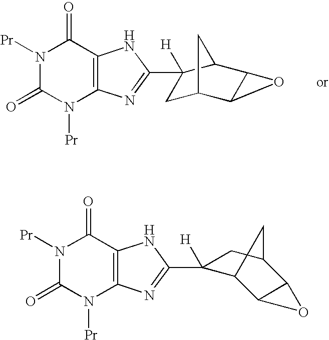

In some embodiments, the compositions described herein can also include a non adenosine-modifying diuretic, such as a proximal, loop, or distal diuretic. For example, in some embodiments, the compositions described herein can include any one or combination of the following non adenosine-modifying diuretics: hydrochlorothiazides, furosemide, torsemide, bumetamide, ethacrynic acid, piretamide, spironolactone, triamterene, norsemide, and amiloridethiazides.

As stated above, embodiments disclosed herein also relate to methods of providing AA₁RA therapy to a patient. The methods disclosed herein can include the step of administering a therapeutically effective amount of an AA₁RA and an effective prophylactic amount of an anticonvulsant to the patient. In some embodiments, the anticonvulsant and the AA₁RA are administered nearly simultaneously. In other embodiments, the administering step comprises administering one of said anticonvulsant and said AA₁RA first and then administering the other one of said anticonvulsant and said AA₁RA. In some embodiments, the subject is identified as being at risk for seizure or convulsions.

For example, some embodiments provide methods for inducing a diuretic effect in a subject, as well as maintaining or restoring the diuretic effect or a non adenosine-modifying diuretic in a subject, by administering a therapeutically effective amount of an AA₁RA and an effective prophylactic amount of an anticonvulsant to the subject. In some embodiments, the subject can be refractory to standard diuretic therapy, whereas in other embodiments, the subject is not refractory to standard diuretic therapy.

Other embodiments relate to treating CHF in a subject administering a therapeutically effective amount of an AA₁RA and an effective prophylactic amount of an anticonvulsant to the subject. In some embodiments, the subject can be refractory to standard diuretic therapy. In other embodiments, the subject is not refractory to standard diuretic therapy. In some embodiments, the subject can have impaired renal function, impaired creatinine clearance, elevated serum creatinine, or any combination thereof. In other embodiments, the subject does not have renal impairment.

Still other embodiments relate to methods of preventing the onset of renal impairment in a subject with fluid overload or CHF, as well as methods of maintaining, restoring, or improving renal function in subjects with CHF. The subject can be administered a therapeutically effective amount of an AA₁RA and an effective prophylactic amount of an anticonvulsant. In some embodiments, the subject can be refractory to standard diuretic therapy. In other embodiments, the subject is not refractory to standard diuretic therapy.

In some embodiments, the subject is administered an amount of an AA₁RA such as KW-3902 to increase the creatinine clearance rate. In some embodiments, the subject is administered an amount of an AA₁RA effective to decrease serum creatinine levels. For example, in some embodiments, patients with impaired creatinine clearance and/or elevated serum creatinine levels are identified and treated according to the methods disclosed herein.

Other embodiments relate to methods of maintaining, restoring or improving renal function in a subject that has been identified with impaired creatinine clearance and/or elevated serum creatinine clearance. The subject can be administered a therapeutically effective amount of an AA₁RA and an effective prophylactic amount of an.

As stated above, in some embodiments, the subject can be administered an effective prophylactic amount of an anticonvulsant. In some embodiments, the anticonvulsant can be selected from one of the following: diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or any combination thereof, or pharmaceutically acceptable salts, prodrugs, esters, or amides, or metabolites thereof. For example, in some embodiments, the anticonvulsant can be diazepam and/or lorazepam.

In some embodiments, the subject can be administered a non adenosine-modifying diuretic in addition to the AA₁RA and anticonvulsant, e.g., a proximal, loop, or distal diuretic. For example, in some embodiments, the methods disclosed herein provide for the administration of one or more of the following non adenosine-modifying diuretics: hydrochlorothiazides, furosemide, torsemide, bumetamide, ethacrynic acid, piretamide, spironolactone, triamterene, and amiloridethiazides. In preferred embodiments, the methods provide for the administration of furosemide.

### BRIEF DESCRIPTION OF THE DRAWINGS

FIG. 1 shows the percentage of subjects treated with 10 mg, 20 mg, 30 mg KW-3902 or placebo falling into the three different categories designated for the primary endpoint of the study described in Example 1: Success, Unchanged, and Failure.

FIG. 2 with acute CHF and mild to severe renal impairment requiring intravenous diuretic therapy shows the change in mean serum creatinine levels over the indicated time period in subjects following treatment with 10 mg, 20 mg, or 30 mg KW-3902, or placebo as described in Example 1.

FIG. 3 shows the percentage of subjects with acute CHF and mild to severe renal impairment requiring intravenous diuretic therapy treated with 10 mg, 20 mg, or mg KW-3902, or placebo that reported moderate or marked improvement in dyspnea, as described in Example 1.

FIG. 4 shows the percentage of subjects with acute CHF and mild to severe renal impairment requiring intravenous diuretic therapy treated with 10 mg, 20 mg, or mg KW-3902, or placebo falling into the “Success” category designated for the primary endpoint of the study over time, as described in Example 1.

FIG. 5 shows the percentage of subjects with acute CHF treated with 10 mg, 20 mg, or 30 mg KW-3902 or placebo exhibiting worsening heart failure over the indicated time periods following treatment, as described in Example 1.

FIG. 6 shows the percentage of subjects with acute CHF treated with 10 mg, 20 mg, or 30 mg KW-3902 or placebo exhibiting worsening renal function over the indicated time periods following treatment, as described in Example 1.

FIG. 7 shows the percentage of subjects with acute CHF treated with 10 mg, 20 mg, or 30 mg KW-3902 or placebo that were discharged from the hospital on days 2 or 3 following treatment, as described in Example 1.

FIG. 8 shows the change in median weight of subjects with acute CHF treated with 10 mg, 20 mg, or 30 mg KW-3902 or placebo on the indicated days following treatment, as described in Example 1.

### DETAILED DESCRIPTION OF THE PREFERRED EMBODIMENT

A significant problem encountered in treating certain conditions with individual medications is that following a course of therapy the patients become refractory to the treatment, i.e., the patients begin to respond less and less to the medication until they do not respond at all. This problem is very common in patients who suffer from, for example, cardiovascular disease, including individuals suffering from congestive heart failure (CHF) who are treated with diuretics.

Individual diuretics act on a specific segment of nephrons, e.g., proximal tubule, loop of Henle, or distal tubule. One mechanism by which diuretics increase urine volume is that they inhibit reabsorption of sodium and accompanying water passing through the nephron. Thus, for example, a loop diuretic inhibits reabsorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium reabsorption elsewhere in the nephron; the other is through feedback via adenosine A₁ receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

AA₁RAs act on the afferent arteriole of the kidney to produce vasodilation and thereby improve renal blood flow in patients with CHF. They also block the TGF mechanism mediated by adenosine (via A₁ receptors) described above. This ultimately allows for increased GFR and improved renal function. In addition, AA₁RAs inhibit the reabsorption of sodium (and, therefore, water) in the proximal tubule, which results in diuresis.

AA₁RAs exert a diuretic effect by inhibiting the reabsorption of sodium in the proximal tubule of the nephron through adenosine A₁ receptors. In addition, AA₁RAs improve renal blood flow and glomerular filtration by inhibiting TGF, which is activated by diuretics that increase distal tubular sodium. Further, it appears that AA₁RAs have anti-oxidant properties in some conditions, such as radiographic contrast-mediated nephropathy, and therefore, may have similar properties in other conditions where oxygen-free radicals are injurious.

Administration of AA₁RAs to individuals in need of diuretics (e.g. presenting with congestive heart failure, diminished renal function, hypertension, asymptomatic left ventricular dysfunction, coronary artery disease, acute myocardial infarction, or suffering from a cardiovascular disease and in need of after-load reduction) may increase the probability of seizures. Not wishing to be bound by any particular mechanism or mode of action and offered only to expand the knowledge in the field, it is contemplated that the administration of AA₁RAs can decrease the seizure threshold in at-risk subjects.

Seizures and convulsions are the consequence of temporary abnormal electrophysiologic phenomena of the brain, resulting in abnormal synchronization of electrical neuronal activity. They can manifest as an alteration in mental state, tonic or clonic movements (discussed below) and various other symptoms. Tonic clonic seizures, also known as grand mal seizures involve two phases: a tonic phase and a clonic phase. The tonic phase involves vocalization, severe hyperextension (opisthotonos), possible respiratory arrest, cyanosis, and reflex bladder emptying. The clonic phase involves rhythmic generalized jerking, followed by prolonged unconsciousness.

Everyone individual has a seizure threshold, i.e., a tolerance point beyond which a seizure can be induced. For example, individuals who develop seizure disorders have a lower threshold for seizures than others. Sleep deprivation, prolonged or acute stress, exhaustion, fear, illness, increases in breathing rates or changes in blood sugar levels are exemplary factors known to lower the seizure threshold.

The term “anticonvulsant,” as used herein, refers to a pharmaceutical useful in the treatment or control of epileptic seizures. The goal of an anticonvulsant is to suppress the rapid and excessive firing of neurons that start a seizure. Anticonvulsants also prevent the spread of the seizure within the brain and offer protection against excitotonic effects that can result in brain damage. Anticonvulsants can exert their anticonvulsive effect through a variety of complex mechanisms of action. Without wishing to be bound to any particular theory, many anticonvulsants are thought to act through one or more of the following mechanisms, involving: 1) mediation of voltage-sensitive ion channels; 2) direct or indirect actions involving a gamma aminobutyric acid (GABA) or the GABAA receptor; and 3) inhibition of excitatory amino acids (e.g., glutamate or aspartate, among others) by acting as an excitatory amino acid (EAA) receptor antagonists. However, some clinically effective anticonvulsant agents do not have a known mechanism of action.

Accordingly, in a first aspect, the invention relates to pharmaceutical compositions that include both an adenosine A₁ receptor antagonist (AA₁RA) and an anticonvulsant.

AA₁RAs

A number of AA₁RAs are known in the art, though currently, none are commercially available as therapeutics. AA₁RAs antagonize the A₁ receptor of adenosine selectively (e.g. they do not substantially antagonize other adenosine receptors). The majority of the known AA₁RAs are derivatives of xanthine and include compounds such as 1,3-dipropyl-8-{3-oxatricyclo[3.1.2.0.^{2,4}]oct-6(7)-yl}xanthine (also known as 1,3-dipropyl-8-[5,6-exo-epoxy-2(S) norbornyl]xanthine, ENX, CVT-124, and BG9928), 8-(3-noradamantyl)-1,3-dipropylxanthine (also known as KW-3902), theophyllilne, and caffeine. Other AA₁RAs are disclosed in U.S. Pat. Nos. 5,446,046, 5,631,260, and 5,668,139, the disclosures of which are all hereby incorporated by reference herein in their entirety, including any drawings. The scope of the present invention includes all those AA₁RAs now known and all those AA₁RAs to be discovered in the future.

KW-3902 is a xanthine-derived adenosine A₁ receptor antagonist (AA₁RA). Its chemical name is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

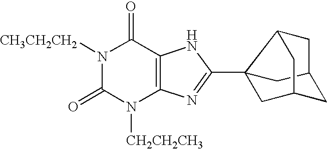

KW-3902 and related compounds useful in the practice of the present invention are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

In some embodiments, the AA₁RA can be a xanthine-derivative compound of Formula I or a pharmaceutically acceptable salt thereof, 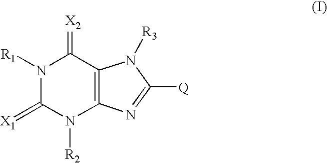
where

each of X₁ and X₂ independently represents oxygen or sulfur;

Q represents: 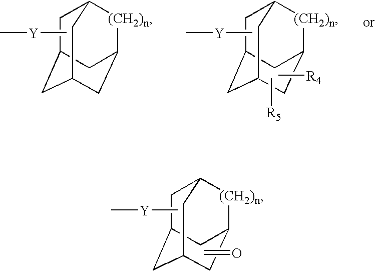
where Y represents a single bond or alkylene having 1 to 4 carbon atoms, n represents 0 or 1;

each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or

R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 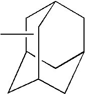
then R₁, R₂ and R₃ are not simultaneously methyl.

In some embodiments, both of R₁ and R₂ of the compound of Formula I are lower alkyl and R₃ is hydrogen; and both of X₁ and X₂ are oxygen. In other embodiments, R₁, R₂ and R₃ independently represents hydrogen or lower alkyl. In still other embodiments, each of R₁ and R₂ independently represents allyl or propargyl and R₃ represents hydrogen or lower alkyl. In certain embodiments, X₁ and X₂ are both oxygen and n is 0.

In some embodiments, R₁ is hydroxy-substituted, oxo-substituted or unsubstituted propyl; R₂ is hydroxy-substituted or unsubstituted propyl; and Y is a single bond. In other embodiments, R₁ is propyl, 2-hydroxypropyl, 2-oxopropyl or 3-oxopropyl; R₂ is propyl, 2-hydroxypropyl or 3-hydroxypropyl.

In some embodiments Q is 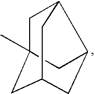
while in other embodiments Q is 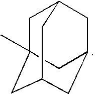
In other embodiments, Q is 9-hydroxy, 9-oxo or 6-hydroxy substituted 3-tricyclo[3.3.1.0^{3,7}]nonyl, or 3-hydroxy-1tricyclo[3.3.1.1^{3,7}]decyl.

In certain embodiments, the AA₁RA is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine (also referred to as “M1-trans”), 8-(cis-9-hydroxy-3-tricyclo[3.3.1.0^{3,7}]nonyl)-1,3-dipropylxanthine (also referred to as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine, or a pharmaceutically acceptable salt thereof.

In other embodiments, the AA₁RA is a xanthine epoxide-derivative compound of Formula II or Formula III, or a pharmaceutically acceptable salt thereof, 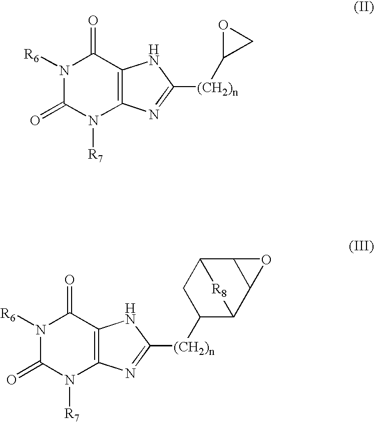
where R₆ and R₇ are the same or different, and can be hydrogen or an alkyl group of 1-4 carbons, R₈ is either oxygen or (CH₂)₁₋₄, and n=0-4.

The xanthine epoxide-derivative compound may be

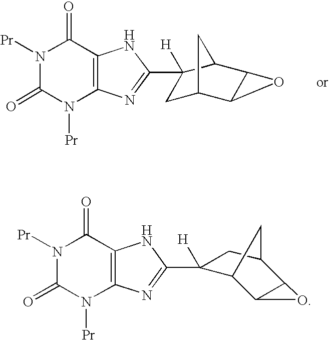

In some embodiments, the AA₁RA is KW-3902. KW-3902 and related compounds useful in the practice of the present invention are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings. Some embodiments provide pharmaceutical compositions that include, or methods that involve administration of KW-3902 in a dose of 2.5 mg, 5 mg, 10 mg, 15 mg, 30 mg, 60 mg, or 100 mg, or higher. In some embodiments, the administered KW-3902 is in an injectable form, while in other embodiments, the administered KW-3902 is in a solid formulation.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which the AA₁RA is converted within the cells of a mammal. The pharmaceutical compositions of the present invention may include a metabolite of KW-3902, or any other AA₁RA instead of KW-3902 or the AA₁RA, respectively. The scope of the methods of the present invention includes those instances where an AA₁RA is administered to the patient, yet the metabolite is the bioactive entity.

Metabolites of KW-3902 are known. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a ═O group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), 8-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine.

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In certain embodiments, the AA₁RA compound described herein is in a formulation described in U.S. Pat. No. 6,210,687, or U.S. Pat. No. 6,254,889, both or which are hereby incorporated by reference herein in their entirety, including any drawings.

Anticonvulsants

Several anticonvulsants are known in the art and are useful in the compositions and methods described herein. See, e.g., U.S. Patent Application Publication No. 2005/0070524 Extensive listings of anticonvulsants can also be found, e.g., in Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083, the disclosures of which are hereby expressly incorporated by reference in their entireties. Non-limiting examples of anticonvulsants that can be used in the compositions and methods disclosed hererin include diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof. However, the inclusion of other anticonvulsants, now known or discovered in the future, is within the scope of the present invention.

The compositions disclosed herein can include anticonvulsants in such amount as will be therapeutically or prophylactically effective in the treatment or control of seizures. It will be appreciated that the amount of anticonvulsant contained in an individual dose of each dosage form of the compositions need not in itself constitute an effective prophylactic amount, as the necessary effective amount could be reached by administration of a number of individual doses. Those skilled in the art will appreciate that the amount of anticonvulsant agent present in the compositions and administered to individuals disclosed herein will vary depending upon the age, sex, and bodyweight of the subject to be treated, the particular method and scheduling of administration, and what other anticonvulsant agent, if any is present in the compositions disclosed herein or administered in the methods disclosed herein. Dosage amounts for an individual patient may thus be above or below the typical dosage ranges. Generally speaking, the anticonvulsant agent can be employed in any amount known to be effective at treating, preventing or controlling seizures. The doses may be single doses or multiple doses per day, with the number of doses taken per day and the time allowed between doses varying depending on the individual needs of the patient. Optimization of treatment, including dosage amount, method and time of administration can be routinely determined by the skilled practitioner. Specific dosage levels for anticonvulsants that can be used in the pharmaceutical compositions and methods described herein, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of which are all hereby expressly incorporated by reference. Representative examples of dosage ranges of anticonvulsants are described below, however, it should be noted that the dosage ranges given below indicate only the typical dosage amounts administered to patients for that particular anticonvulsant agent for the treatment of seizures or epilepsy. Thus they should not be construed as limiting amounts for the purpose of the present invention, as actual therapeutically effective dosage amounts for a patient may be more or less than the exemplary dosage range, depending on the individual.

The following section describes in further detail several anticonvulsants useful in the compositions and methods described herein. Accordingly, pharmaceutical compositions that include and AA₁RA and any of the anticonvulsant agents below are contemplated. Further, methods of treating cardiovascular disease by administering an AA₁RA and any of the anticonvulsant agents below are contemplated. The skilled artisan will appreciate that pharmaceutical compositions disclosed herein are preferably formulated to deliver an appropriate amount of an anticonvulsant agent based on the desired dosage and number of dosages. Further, the skilled artisan will appreciate that the methods disclosed herein preferably include administration of an appropriate amount of an anticonvulsant agent, such as for example by the routes and dosage intervals described below.

Diazepam

By way of example, when the anticonvulsant agent is diazepam, it is typical when administered orally, the amount used is within the range of approximately 4 to 40 mg/day, usually in 2 to 4 doses. When administered parenterally, the amount used is typically within the range of approximately 5 to 30 mg. The dose may be repeated, but usually is not more than 600 mg/day.

Midazolam

In another example, when the anticonvulsant agent is midazolam, it is typical that the amount used is within the range of approximately 0.07-0.08 mg/kg/day (approximately 5 mg).

Phenylion/Fosphenyloin

By way of example, when the anticonvulsant agent is phenyloin or fosphenytion, it is typical that the amount used is within the range of approximately 200 to 600 mg/day. The initial dose is typically within the range of approximately 3 to 5 mg/kg (200 to 400 mg/day) in 2 to 3 divided doses if given orally or approximately 10 to 20 mg/kg in one dose if given parenterally.

Phenobarbital

By way of example, when the anticonvulsant agent is Phenobarbital, it is typical that when administered orally, the amount used is within the range of approximately 30 to 320 mg/day. When administered parenterally, the amount used is typically within the range of approximately 100 to 320 mg/. The dose may be repeated, but usually is not more than 600 mg/day.

Mysoline

In another example, when the anticonvulsant agent is mysoline, it is typical that the amount used is within the range of 100 to 125 mg/day, but usually not more than 2000 mg/day.

Clonazepam

In another example, when the anticonvulsant agent is clonazepam, it is typically that the amount used is within the range of approximately 0.5 to 20 mg/day in 2 to 4 divided doses, with the initial dose typically being approximately 1.5 mg/day.

Clorazepate

By way of example, when the anticonvulsant agent is clorazepate, it is typical that the amount used is within the range of approximately 15 to 90 mg/day in 1 to 4 divided doses, with the initial dose typically ibeing approximately 7.5 to 22.5 mg/day.

Carbamazepine

By way of example, when the anticonvulsant agent is carbamazepine, it is typical that the amount used is within the range of approximately 400 to 2400 mg/day divided into 2 to 4 doses, with the initial dose typically being approximately 100 to 200 mg taken 1 to 2 times per day. Optimal dosage amounts will vary depending on the needs of the individual patient, but preferably will not exceed 1200 mg/day. Dosages may be administered one to four times per day, depending on the needs of the individual patient and the dosage form. Typically, a low initial dose with a gradual increase to the minimum effective dose is advised.

Oxcarbazepine

In a further example, when the anticonvulsant agent is oxcarbazepine, it is typical that the amount used is within the range of approximately 900 to 3000 mg/day with the initial dose typically being approximately 400 to 600 mg/day in two divided doses.

Valproic Acid/Sodium Valproate

In a further example, when the anticonvulsant agent is valproic acid, sodium valproate, or derivatives thereof, dose can be based on body weight. Typically the amount used is within the range of approximately 10 to 60 mg/kg/day (or 375 to 4000 mg/day) given in 2 to 4 divided doses, with the initial dose typically being approximately 5 to 30 mg/kg/day (or 250 to 750 mg/day) given at 2 to 4 divided doses. The initial dose is then typically gradually increased by 5 to 10 mg/kg/week, as needed.

Gabapentin

In a further example, when the anticonvulsant agent is gabapentin, it is typical that that amount used is within the range of approximately 600 to 4800 mg/day, with the initial does typically being approximately 300 to 900 mg/day. The total daily dose is typically divided, and administered in 3 to 4 doses per day.

Topiramate

In another example, when the anticonvulsant agent is topiramate, it is typical that the amount used is within the range of approximately 200 to 400 mg/day, with the initial dose typically being approximately 25 to 50 mg/day and slowly adjusted upwards as needed. Doses are typically divided and administered twice daily.

Felbamate

In a further example, when the anticonvulsant agent is felbamate, it is typical that the amount used is within the range of approximately 600 to 3600 mg/day in 3 to 4 divided doses, with the initial dose typically being approximately 600 to 1200 mg/day.

Tiagabin

In a further example, when the anticonvulsant agent is tiagabin, it is typical that the amount used is within the range of approximately 32 to 64 mg/day, with the initial dose typically being approximately 4 mg/day. Doses are typically divided and given 2 to 4 times daily.

Lamotrigine

In yet another example, when the anticonvulsant agent is lamotrigine, the amount administered will vary depending on patient age and what other anticonvlulsant agents, if any, are co-administered. The amount of lamotrigine administered is typically within the range 25 mg every other day to 700 mg/day. The amount of lamotrigine administed to patient who are also taking enzyme-inducing anticonvulsant agents (e.g. carbamazepine, Phenobarbital, phenyloin, and/or primidone) but are not taking valproic acid (or derivatives) is preferably within the range of approximately 50 to 500 mg/day. The amount of lamotrigine administered to patients who are taking enzyme inducing anticonvulsant agents and valproic acid is preferably within the range of approximately 25 mg every other day to 400 mg/day. Initial doses are usually within the lower end of the dosage ranges, and can be increased slowly, as needed, to avoid side effects.

Famotidine

In still another example, when the anticonvulsant agent is famotidine, it is typical that the amount administered is approximately 20 mg/day, or more, but does not typically exceed 640 mg/day.

Ethotoin

In yet another example, when the anticonvulsant agent is ethotoin, it is typical that the amount used is within the range of approximately 125 to 250 milligrams 4 to 6 times a day. Typically, the dose usually does not exceed more than 3000 mg a day.

Mephobarbital

In another example, when the anticonvulsant agent is mephobarbital, it is typical that the amount used is within the range of approximately 32-100 mg 3 to 4 times a day, or 200 to 600 mg day in 3 to 4 divided doses.

Metharbital

In another example, when the anticonvulsant agent is metharbital, it is typical that the amount used is within the range of approximately 100-300 mg a day, in 1 to 3 divided doses. Typically, the dose usually does not exceed more than 800 mg/day.

Ethosuximide

In yet another example, when the anticonvulsant agent is ethosuximide, it is typical that the amount used is within the range of approximately 500 to 2000 mg/day, with the initial dose typically being approximately 250 to 500 mg/day. The total daily dose may be administered in one daily dose, or divided and given in two doses per day.

Methsuximide

In yet another example, when the anticonvulsant agent is methsuximide, it is typical that the amount used is within the range of approximately 300 to 1200 mg/day.

Trimethadione/Paramethadione

In yet another example, when the anticonvulsant agent is trimethadione or paramethadione, it is typical that the amount used is within the range of approximately 300 mg 3 to 4 times per day. Typically, the daily dose will not exceed 2400 mg/day, divided in 3 to 4 doses.

Phenacemaide

In yet another example, when the anticonvulsant agent is Phenacemide, it is typical that the amount used is within the range of approximately 500 mg three times a day. Typically, the daily dose will not exceed 5000 mg/day.

Acetozolamide

In yet another example, when the anticonvulsant agent is acetozolamide, it is typical that the amount used is within the range of approximately 10 mg/kg/day in 1-3 divided doses.

Clobazam

In yet another example, when the anticonvulsant agent is clobazam, it is typical that the amount used is within the range of approximately 500 to 2000 mg/day, with the initial dose typically being approximately 250 to 500 mg/day. The total daily dose may be administered in one daily dose, or divided and given in two doses per day.

Levetiracetam

In a further example, when the anticonvulsant agent is levetiractam, it is typical that the amount used is within the range of approximately 1000 to 3000 mg/day, with the initial dose typically being approximately 1000 mg/day in two divided doses.

Primidone

In another example, when the anticonvulsant agent is primidone, it is typical that the amount used is within the range of approximately 250 to 2000 mg/day in divided doses, with the initial dose typically being approximately 100 to 125 mg/day.

Lorazepam

In another example, when the anticonvulsant agent is lorazepam, it is typical that administration is by intravenous bolus injection, at approximately 0.07 mg/kg (to a maximum of 4 mg) is given, and this can be repeated once after 20 minutes if no effect has been observed.

Thiopentone

In a further example, when the anticonvulsant agent is thiopentone, it is typical that the amount used is 100-250 mg initially in a bolus injection, with further 50 mg boluses every 2-3 minutes until seizures are controlled. Intravenous infusion is then typically continued at between 3 and 5 mg/kg per hour, and at thiopentone blood levels of about 40 mg/l.

Propofol

In a still further example, when the anticonvulsant agent is propofol, it is typical tht the amount used is within the range of 1-2 mg/kg initially as a bolus dose, which can be repeated if seizures continue, succeeded by an infusion of 1-15 mg/kg per hour.

Zonisamide

In another example, when the anticonvulsant agent is zonisamide, it is typical that the amount used is within the range of approximately 100 to 8000 mg/day in two divided doses, with the initial dose typically being approximately 100 mg/day in one dose.

In some embodiments, the composition can include more than one anticonvulsant and an AA₁RA. For example, the composition can include 2 or 3 or more anticonvulsant agents.

Combinations with Non-AA₁RA Diuretics

In some embodiments, the pharmaceutical compositions can also include a non-adenosine modifying diuretic. In some embodiments, the non-adenosine modifying diuretic is a proximal diuretic, i.e., a diuretic that principally acts on the proximal tubule. Examples of proximal diuretics include, but are not limited to, acetazolamide, methazolamide, and dichlorphenamide. Carbonic anhydrase inhibitors are known to be diuretics that act on the proximal tubule, and are therefore, proximal diuretics. Thus, some embodiments provide compositions that include the combination of an AA₁RA (e.g., KW-3902), and an anticonvulsant agent with a carbonic anhydrase inhibitor. Combinations of an AA₁RA (e.g., KW-3902), and an anticonvulsant agent with any proximal diuretic now known or later discovered are within the scope of the embodiments disclosed herein.

In other embodiments, the non-adenosine modifying diuretic is a loop diuretic, i.e., a diuretic that principally acts on the loop of Henle. Examples of loop diuretics include, but are not limited to, furosemide (LASIX®), bumetamide (BUMEX®), and torsemide (TOREM®). Combinations of an AA₁RA and an anticonvulsant agent with any loop diuretic now known or later discovered are within the scope of the embodiments disclosed herein.

In yet other embodiments, the non-adenosine modifying diuretic is a distal diuretic, i.e., a diuretic that principally acts on the distal nephron. Examples of distal diuretics include, but are not limited to, metolazone, thiazides and amiloride. Combinations of an AA₁RA and an anticonvulsant agent with any distal diuretic now known or later discovered are within the scope of the embodiments disclosed herein.

Combinations with Beta-Blockers

In some embodiments, the pharmaceutical compositions can also include a beta-blocker. A number of beta-blockers are commercially available. These compounds include, but are not limited to, acebutolol hydrochloride, atenolol, betaxolol hydrochloride, bisoprolol fumarate, carteolol hydrochloride, esmolol hydrochloride, metoprolol, metoprolol tartrate, nadolol, penbutolol sulfate, pindolol, propranolol hydrochloride, succinate, and timolol maleate. Beta-blockers, generally, are beta₁ and/or beta₂ adrenergic receptor blocking agents, which decrease the positive chronotropic, positive inotropic, bronchodilator, and vasodilator responses caused by beta-adrenergic receptor agonists. The scope of the present invention includes all those beta-blockers now known and all those beta-blockers to be discovered in the future.

Combinations with Angiotensin Converting Enzyme Inhibitors or Angiotensin II Receptor Blockers

In some embodiments, the pharmaceutical compositions can also include an angiotensin converting enzyme inhibitor or an angiotensin II receptor blocker. A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. The scope of the present invention includes all those ACE inhibitors now known and all those ACE inhibitors to be discovered in the future.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs function stems from their ability to block the binding of angiotensin II, which would normally cause vessels to constrict. The scope of the present invention includes all those ARBs now known and all those ARBs to be discovered in the future.

Other aspects relate to methods of treating patients using a therapeutically effective amount of an AA₁RA, or a salt, ester, amide, metabolite, or prodrug thereof, and an anticonvulsant agent. Yet another aspect relates to methods of improving diuresis while maintaining renal function in individuals with fluid overload, using a therapeutically effective amount of an AA₁RA, (e.g. KW-3902), or a salt, ester, amide, metabolite, or prodrug thereof, and an anticonvulsant agent. In certain embodiments, the individual being treated by the methods of the present invention suffers from renal impairment. In other embodiments, the individual does not suffer from renal impairment. These individuals include those who suffer from heart failure, such as congestive heart failure, or other maladies that result in fluid overload, without having yet disrupted normal kidney function. In some embodiments, the individual being treated by the methods of the present invention is refractory to standard diuretic therapy. In other embodiments, the individual is not refractory to standard diuretic therapy.

The compositions and methods described herein act to induce a diuretic effect in an animal, while reducing the potential of related adverse events occurring, such as seizures or convulsions. Accordingly, in another aspect, embodiments herein relate to methods of comprising identifying a patient in need thereof and administering to the patient a therapeutically effective amount of an AA₁RA (e.g. KW-3902), in combination with an anticonvulsant agent.

In certain embodiments, the patient may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the patient is a human.

In another aspect, the present invention relates to a method of maintaining or restoring the diuretic effect of a non-adenosine modifying diuretic in a patient, while reducing the potential of related adverse events occurring, such as seizures or convulsions, comprising identifying a patient in need thereof, and administering to the patient a therapeutically effective amount of an AA₁RA, (e.g., KW-3902), or a pharmaceutically acceptable salt, ester, amide, metabolite, or prodrug thereof, in combination with a non-adenosine modifying diuretic and an anticonvulsant agent. For example some embodiments relate to methods of maintaining or restoring the diueritic effect of a diuretic such as furosemide. In some embodiments, furosemide is administered in a dose of 20 mg, 40 mg, 60 mg, 80 mg, 100 mg, 120 mg, 140 mg, or 160 mg, or higher. The administration may be oral or intravenous. When furosemide is administered intravenous, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

In yet another aspect, the present invention relates to a method of maintaining or restoring renal function in a patient comprising identifying a patient in need thereof, and administering a therapeutically effective amount of an AA₁RA (e.g., KW-3902), or a pharmaceutically acceptable salt, ester, amide, metabolite, or prodrug thereof, in combination with an anticonvulsant agent and a second pharmaceutical composition capable of inducing a diuretic effect.

In the context of the present disclosure, by “maintaining” renal function it is meant that the renal function, as measured by creatinine clearance rate, remains unchanged for a period of time after the start of the therapy. In other words, by “maintaining” renal function it is meant that the rate of renal impairment, i.e., the rate of decrease in the creatinine clearance rate, is slowed or arrested for a period of time, however brief that period may be. By “restoring” renal function it is meant that the renal function, as measured by creatinine clearance rate, has improved, i.e., has become higher, after the start of the therapy.

In a further aspect, the present invention relates to a method of treating a patient with a pharmaceutical composition as described herein. In some embodiments, the patient is refractory to standard diuretic therapy.

Certain patients who suffer from a cardiac condition, such as congestive heart failure, later develop renal impairment. The present inventors have discovered that if a patient presented with a cardiac condition, and little to no renal impairment, is treated with a pharmaceutical composition as described herein, the onset of renal impairment is delayed or arrested, compared to a patient who receives standard treatment. Thus, aspects of the present invention relate to a method of preventing the deterioration of renal function, delaying the onset of renal impairment, or arresting the progress of renal impairment in a patient comprising identifying a patient in need thereof, and administering a therapeutically effective amount of an AA₁RA (e.g. KW-3902), or a salt, ester, amide, metabolite, or prodrug thereof, and an anticonvulsant agent and a non-adenosine modifying diuretic.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved. Thus, in the context of the present invention, increasing the urine output volume, decreasing the level of serum creatinine, or increasing creatinine clearance, may be considered treatment, even if the patient is not cured or does not generally feel better.

In another aspect, the present invention relates to a method of treating a patient suffering from CHF comprising identifying a patient in need thereof, and administering to said patient a therapeutically effective amount of amount of an AA₁RA (e.g., KW-3902), or a salt, ester, amide, metabolite, or prodrug thereof, and an anticonvulsant agent and a non-adenosine modifying diuretic.

In a further aspect, the present invention relates to a method of improving overall health outcomes, decreasing morbidity rates, or decreasing mortality rates in patients comprising identifying a patient in need thereof, and administering to said patient a therapeutically effective amount of amount of an AA₁RA (e.g., KW-3902), or a salt, ester, amide, metabolite, or prodrug thereof, and an anticonvulsant agent and a non-adenosine modifying diuretic.

Overall health outcomes are determined by various means in the art. For example, improvements in morbidity and/or mortality rates, improvements in the patient's general feelings, improvements in the quality of life, improvements in the level of comfort at the end of life, and the like, are considered when overall health outcome are determined. Mortality rate is the number of patients who die while undergoing a particular treatment for a period of time compared to the overall number of patients undergoing the same or similar treatment over the same period of time. Morbidity rates are determined using various criteria, such as the frequency of hospital stays, the length of hospital stays, the frequency of visits to the doctor's office, the dosage of the medication being administered, and the like.

In yet another aspect, the methods of the present invention relate to the prevention of the deterioration of renal function in individuals comprising administering a therapeutically effective amount of KW-3902, or a salt, ester, amide, metabolite, or prodrug thereof, and an anticonvulsant. In some embodiments, the method also includes that administration of a non-adenosine modifying diuretic.

In some embodiments, the patient whose overall health outcome, morbidity and/or mortality rate is being improved suffers from CHF. In other embodiments, the patient suffers from renal impairment.

In some embodiments, the administering step comprises administering said anticonvlusant and said AA₁RA nearly simultaneously. These embodiments include those in which the AA₁RA and the anticonvulsant are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments the administering step comprises administering the anticonvulsant first and then administering the AA₁RA (e.g., KW-3902). In yet other embodiments, the administering step comprises administering the AA₁RA (e.g., KW-3902), first, and then administering the anticonvulsant. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally.

The methods of the present invention are intended to provide treatment for cardiovascular disease, which may include congestive heart failure, hypertension, asymptomatic left ventricular dysfunction, coronary artery disease, or acute myocardial infarction. In some instances, patients suffering from a cardiovascular disease are in need of after-load reduction. The methods of the present invention are suitable to provide treatment for these patients as well. Certain patients who suffer from a cardiac condition, such as congestive heart failure, later develop renal impairment.

In another aspect, the invention relates to the treatment of cardiovascular diseases using a combination of a beta-blocker, an AA₁RA and an anticonvulsant agent. The present inventors have discovered that the combination of AA₁RAs and beta blockers is beneficial in either congestive heart failure (CHF) or hypertension, or any of the other indications set forth herein. See, co-pending U.S. application Ser. No. 10/785,446 entitled “Method of Treatment of Disease Using and Adenosine A1 Receptor Antagonist,” filed Feb. 23, 2004, herein expressly incorporated by reference in its entirety.

Beta-blockers are known to have antihypertensive effects. While the exact mechanism of their action is unknown, possible mechanisms, such as reduction in cardiac output, reduction in plasma renin activity, and a central nervous system sympatholytic action, have been put forward. From various clinical studies, it is clear that administration of beta-blockers to patients with hypertension results initially in a decrease in cardiac output, little immediate change in blood pressure, and an increase in calculated peripheral resistance. With continued administration, blood pressure decreases within a few days, cardiac output remains reduced, and peripheral resistance falls toward pretreatment levels. Plasma renin activity is also reduced markedly in patients with hypertension, which will have an inhibitory action on the renin-angiotensin system, thus decreasing the after-load and allowing for more efficient forward function of the heart. The use of these compounds has been shown to increase survival rates among patients suffering from CHF or hypertension. The compounds are now part of the standard of care for CHF and hypertension. The combination of an AA₁RA (e.g., KW-3902), a beta blocker, and an anticonvulsant agent acts synergistically to further improve the condition of patients with hypertension or CHF while reducing the potential of related adverse events occurring, such as seizures or convulsions. The diuretic effect of AA₁RAs, especially in salt-sensitive hypertensive patients along with the blockage of beta adrenergic receptors decreases blood pressure through two different mechanisms, whose effects build on one another. In addition, most CHF patients are also on additional diuretics. The combination allows for greater efficacy of other more distally acting diuretics by improving renal blood flow and renal function.

Beta-blockers are well established in the treatment of hypertension. The addition of AA₁RAs will further treat hypertension via its diuretic effect from inhibiting sodium reabsorption through the proximal tubule. In addition, since many hypertensive patients are sodium sensitive, the addition of an AA₁RA to a beta-blocker will result in further blood pressure reduction. AA₁RA action on tubuloglomerular feedback further improves renal function to result in greater diuresis and lower blood pressure.

In some embodiments concerning methods involving the administration of an AA₁RA, a beta blocker, and an anticonvulsant agent, the administering step comprises administering said beta-blocker, said AA₁RA, and said anticonvulsant agent nearly simultaneously. These embodiments include those in which the AA₁RA, the beta-blocker are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments the administering step comprises administering one of the beta-blocker and the AA₁RA first and then administering the other one of the beta-blocker and the AA₁RA. In these embodiments, the patient may be administered a composition comprising one or more of the compounds and then at some time, a few minutes or a few hours, later be administered another composition comprising the other one or more of the remaining compounds. Also included in these embodiments are those in which the patient is administered a composition comprising one of the compounds on a routine or continuous basis while receiving a composition comprising the other compound occasionally.

In another aspect, the invention relates to the treatment of renal and/or cardiac diseases using a combination of an AA₁RA, an anticonvulsant agent, and an angiotensin converting enzyme (ACE) inhibitor or an angiotensin II receptor blocker (ARB). AA1RAs, ACE inhibitors and ARBs have individually been shown to be somewhat effective in the treatment of cardiac disease, such as congestive heart failure, hypertension, asymptomatic left ventricular dysfunction, or acute myocardial infarction, or renal disease, such as diabetic nephropathy, contrast-mediated nephropathy, toxin-induced renal injury, or oxygen free-radical mediated nephropathy.

The present inventors have discovered that the combination of AA₁RAs and ACE inhibitors or ARBs is beneficial in either congestive heart failure (CHF) or hypertension. See, co-pending U.S. application Ser. No. 10/785,446. The use of ACE inhibitors and ARBs in CHF relies on inhibition of renin-angiotensin system. These compounds decrease the after-load, thereby allowing for more efficient forward function of the heart. In addition, renal function is “normalized” or improved such that patients remove excess fluid more effectively. The use of these compounds has been shown to increase survival rates among patients suffering from CHF or hypertension. The compounds are now part of the standard of care for CHF and hypertension.

The combination of AA₁RAs and ACE inhibitors or ARBs acts synergistically to further improve renal function for continued diuresis. In addition, most CHF patients are also on additional diuretics. The combination allows for greater efficacy of other more distally acting diuretics by improving renal blood flow and renal function.

Both ACE inhibitors and ARBs are well established in the treatment of hypertension via their action through the renin-angiotensin system. The addition of AA₁RAs will further treat hypertension via its diuretic effect from inhibiting sodium reabsorption through the proximal tubule. In addition, since many hypertensive patients are sodium sensitive, the addition of an AA₁RA to an ACE inhibitor or an ARB will result in further blood pressure reduction. AA₁RA action on tubuloglomerular feedback further improves renal function to result in greater diuresis and lower blood pressure.

ACE inhibitors and ARBs are also known to prevent some of the renal damage induced by the immunosuppresant, cyclosporin A. However, there is a renal damaging effect despite their use. The present inventors have discovered that the combination ACE inhibitors and ARBs with AA₁RAs would be more effective in preventing drug-induced nephrotoxicity, such as that induced by cyclosporin A, contrast medium (iodinated), and aminoglycoside antibiotics. In this setting there is renal vasoconstriction that can be minimized by both compounds. In addition, direct negative effects on the tubular epithelium by cyclosporin is less prominent in the setting of adenosine A₁ receptor antagonism, in that blocking A₁ receptors decreases active processes. Furthermore, there are fewer oxidative by-products that are injurious to the tubular epithelium. In addition, the inhibitory effect of AA₁RA blockade on the tubuloglomerular feedback mechanism helps preserve function in the setting of nephrotoxic drugs.

It is known that ACE inhibitors and ARBs are beneficial in preventing the worsening of renal dysfunction in diabetics as measured by albuminuria (proteinuria). Once diabetes begins, glucosuria develops and the kidneys begin to actively reabsorb glucose, especially through the proximal convoluted tubule. This active process may result in oxidative stress and begin the disease process of diabetic nephropathy. Early manifestations of this process are hypertrophy and hyperplasia of the kidney. Ultimately, the kidney begins to manifest other signs such as microalbuminuria and decreased function. It is postulated that the active reabsorption of glucose is mediated in part by adenosine A₁ receptors. Blockade of this process by an AA₁RA limits or prevents the early damage manifested in diabetics.

The combination of AA₁RA and ACE inhibitors or ARBs, as disclosed herein, works to limit both early and subsequent damage to the kidneys in diabetes. The presently disclosed combinations are given at the time of diagnosis of diabetes or as soon as glycosuria is detected in at risk patients (metabolic syndrome). The long-term treatment using the combinations of the present invention includes daily administration of the pharmaceutical compositions described herein.

In another aspect, the invention relates to a method of treating cardiovascular disease or renal disease comprising identifying a patient in need of such treatment, and administering a combination of an AA₁RA, an anticonvulsant agent, and an angiotensin converting enzyme (ACE) inhibitor or an angiotensin II receptor blocker (ARB) to said patient. In certain embodiments, the patient may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the patient is a human.

In some embodiments, the administering step comprises administering said ACE inhibitor or said ARB and said AA₁RA and anticonvulsant nearly simultaneously. These embodiments include those in which the AA₁RA, anticonvulsant and the ACE inhibitor or ARB are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments the administering step comprises administering one of the ACE inhibitor or ARB and the AA₁RA first and then administering the other one of the ACE inhibitor or ARB and the AA₁RA. The anticonvulsant agent can be administered either before, after, or simultaneously with any of the either the ACE inhibitor or ARB or the AA₁RA. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally.

The methods of the present invention are intended to provide treatment for cardiovascular disease, which may include congestive heart failure, hypertension, asymptomatic left ventricular dysfunction, or acute myocardial infarction. In some instances, patients suffering from a cardiovascular disease are in need of after-load reduction. The methods of the present invention are suitable to provide treatment for these patients as well.

The methods of the present invention are also intended to provide treatment for renal disease, which may include renal hypertrophy, renal hyperplasia, microproteinuria, proteinuria, diabetic nephropathy, contrast-mediated nephropathy, toxin-induced renal injury, or oxygen free-radical mediated nephropathyhypertensive nephropathy, diabetic nephropathy, contrast-mediated nephropathy, toxin-induced renal injury, or oxygen free-radical mediated nephropathy.

Still other aspects relate to methods of treating alkosis using an AA₁RA and anticonvulsant. Alkalosis is an acid-base disturbance caused by an elevation in plasma bicarbonate (HCO₃⁻) concentration. It is a primary pathophysiologic event characterized by the gain of bicarbonate or the loss of nonvolatile acid from extracellular fluid. The kidney preserves normal acid-base balance by two mechanisms: bicarbonate reclamation, mainly in the proximal tubule, and bicarbonate generation, predominantly in the distal nephron. Bicarbonate reclamation is mediated mainly by a Na⁺—H⁺ antiporter and to a smaller extent by the H⁺-ATPase (adenosine triphosphatase). The principal factors affecting HCO₃⁻ reabsorption include effective arterial blood volume, glomerular filtration rate, potassium, and partial pressure of carbon dioxide. Bicarbonate regeneration is primarily affected by distal Na⁺ delivery and reabsorption, aldosterone, systemic pH, ammonium excretion, and excretion of titratable acid.

There are a number of different types of alkalosis, for instance metabolic alkalosis and respiratory alkalosis. Respiratory alkalosis is a condition that affects mountain climbers in high altitude situations.

To generate metabolic alkalosis, either a gain of base or a loss of acid must occur. The loss of acid may be via the upper gastrointestinal tract or via the kidney. Excess base may be gained by oral or parenteral HCO₃⁻ administration or by lactate, acetate, or citrate administration.

Factors that help maintain metabolic alkalosis include decreased glomerular filtration rate, volume contraction, hypokalemia, and aldosterone excess. Clinical states associated with metabolic alkalosis are vomiting, mineralocorticoid excess, the adrenogenital syndrome, licorice ingestion, diuretic administration, and Bartter's and Gitelman's syndromes.

The two types of metabolic alkalosis (i.e., chloride-responsive, chloride-resistant) are classified based upon the amount of chloride in the urine. Chloride-responsive metabolic alkalosis involves urine chloride levels less than 10 mEq/L, and it is characterized by decreased extracellular fluid (ECF) volume and low serum chloride such as occurs with vomiting. This type responds to administration of chloride salt. Chloride-resistant metabolic alkalosis involves urine chloride levels more than 20 mEq/L, and it is characterized by increased ECF volume. As the name implies, this type resists administration of chloride salt. Ingestion of excessive oral alkali (usually milk plus calcium carbonate) and alkalosis complicating primary hyperaldosteronism are examples of chloride resistant alkalosis.

Many patients with edematous states are treated with diuretics. Unfortunately, with continued therapy, the patient's bicarbonate level increases and progressive alkalosis may ensue. Diuretics cause metabolic alkalosis by several mechanisms, including (1) acute contraction of the extracellular fluid (ECF) volume (NaCl excretion without HCO₃⁻), thereby increasing the concentration of HCO₃⁻ in the ECF; (2) diuretic-induced potassium and chloride depletion; and (3) secondary aldosteronism. Continued use of the diuretic or either of the latter two factors will maintain the alkalosis.

The addition of an AA₁RA allows continued diuresis and maintained renal function without worsening the alkalosis. The AA₁RA inhibits the active resorption of HCO₃⁻ across the proximal tubule of the kidney.

Thus, in one aspect, the present invention relates to a method of treating metabolic alkalosis while reducing the potential of related adverse events occurring, such as seizures or convulsions, comprising identifying a patient in need thereof and administering a an adenosine A₁ receptor antagonist (AA₁RA) and an anticonvulsant agent to said patient. In certain embodiments, the patient is suffering from high altitude mountain sickness. In some embodiments, the patient has edema. In some of these embodiments, the patient may be on diuretic therapy. The diuretic may be a loop diuretic, proximal diuretic, or distal diuretic. In other embodiments, the patient suffers from acid loss through the patient's upper gastrointestinal tract, for example, through excessive vomiting. In still other embodiments the patient has ingested excessive oral alkali. The methods of the present invention can be practiced with any compound that antagonizes adenosine A₁ receptors.

Yet another aspect relates to the treatment of diabetic neuropathy with an AA₁RA and an anticonvulsant agent. Uncontrolled diabetes causes damage to many tissues of the body. Kidney damage caused by diabetes most often involves thickening and hardening (sclerosis) of the internal kidney structures, particularly the glomerulus (kidney membrane). Kimmelstiel-Wilson disease is the unique microscopic characteristic of diabetic nephropathy in which sclerosis of the glomeruli is accompanied by nodular deposits of hyaline.

The glomeruli are the site where blood is filtered and urine is formed. They act as a selective membrane, allowing some substances to be excreted in the urine and other substances to remain in the body. As diabetic nephropathy progresses, increasing numbers of glomeruli are destroyed, resulting in impaired kidney functioning. Filtration slows and protein, namely albumin, which is normally retained in the body, may leak in the urine. Albumin may appear in the urine for 5 to 10 years before other symptoms develop. Hypertension often accompanies diabetic nephropathy.

Diabetic nephropathy may eventually lead to the nephrotic syndrome (a group of symptoms characterized by excessive loss of protein in the urine) and chronic renal failure. The disorder continues to progress, with end-stage renal disease developing, usually within 2 to 6 years after the appearance of renal insufficiency with proteinuria.

The mechanism that causes diabetic nephropathy is unknown. It may be caused by inappropriate incorporation of glucose molecules into the structures of the basement membrane and the tissues of the glomerulus. Hyperfiltration (increased urine production) associated with high blood sugar levels may be an additional mechanism of disease development.

The diabetic nephropathy is the most common cause of chronic renal failure and end stage renal disease in the United States. About 40% of people with insulin-dependent diabetes will eventually develop end-stage renal disease. 80% of people with diabetic nephropathy as a result of insulin-dependent diabetes mellitus (IDDM) have had this diabetes for 18 or more years. At least 20% of people with non-insulin-dependent diabetes mellitus (NIDDM) will develop diabetic nephropathy, but the time course of development of the disorder is much more variable than in IDDM. The risk is related to the control of the blood-glucose levels. Risk is higher if glucose is poorly controlled than if the glucose level is well controlled.

Diabetic nephropathy is generally accompanied by other diabetic complications including hypertension, retinopathy, and vascular (blood vessel) changes, although these may not be obvious during the early stages of nephropathy. Nephropathy may be present for many years before nephrotic syndrome or chronic renal failure develops. Nephropathy is often diagnosed when routine urinalysis shows protein in the urine.

Current treatments for diabetic nephropathy include administration of angiotensin converting enzyme inhibitors (ACE Inhibitors) during the more advanced stages of the disease. Currently there is no treatment in the earlier stages of the disease since ACE inhibitors may not be effective when the disease is symptom-free (i.e., when the patient only shows proteinuria).

Although the mechanism implicated in early renal disease in diabetics is that of hyperglycemia, a potential mechanism may be related to the active reabsorption of glucose in the proximal tubule. This reabsorption is dependent in part on adenosine A₁ receptors.

AA₁RAs act on the afferent arteriole of the kidney to produce vasodilation and thereby improve renal blood flow in patients with diabetes. This ultimately allows for increased GFR and improved renal function. In addition, AA₁RAs inhibit the reabsorption of glucose in the proximal tubule in patients with newly diagnosed diabetic mellitus or in patients at risk for the condition (metabolic syndrome).

Thus, in one aspect, the present invention relates to a method of treating diabetic nephropathy while reducing the potential of related adverse events occurring, such as seizures or convulsions, comprising identifying a patient in need thereof and administering a an adenosine A₁ receptor antagonist (AA₁RA) and an anticonvulsant to said patient. In certain embodiments the patient is pre-diabetic, whereas in other embodiments the patient is in early stage diabetes. In some embodiments the patient suffers from insulin-dependent diabetes mellitus (IDDM), whereas in other embodiments the patient suffers from non-insulin-dependent diabetes mellitus (NIDDM).

In certain embodiments, the methods of the present invention are used to prevent or reverse renal hypertrophy. In other embodiments, the methods of the present invention are used to prevent or reverse renal hyperplasia. In still other embodiments, the methods of the present invention are used to ameliorate microproteinuria or proteinuria.

Before people develop type II diabetes, i.e., NIDDM, they almost always have “pre-diabetes.” Pre-diabetic patients have blood glucose levels that are higher than normal but not yet high enough to be diagnosed as diabetes. For instance, the blood glucose level of pre-diabetic patients is between 110-126 mg/dL, using the fasting plasma glucose test (FPG), or between 140-200 mg/dL using the oral glucose tolerance test (OGTT). Blood glucose levels below 110 or 140, using FPG or OGTT, respectively, is considered normal, whereas individuals with blood glucose levels higher than 126 or 200, using FPG or OGTT, respectively, are considered diabetic. The methods of the present invention can be practiced with any compound that antagonizes adenosine A₁ receptors.

In certain aspects, the methods of the present invention can be practiced using a combination therapy, i.e., where the AA₁RA and anticonvulsant are administered to the patient in combination with a second compound. In certain embodiments the second compound may be selected from a protein kinase C inhibitor, an inhibitor of tissue proliferation, an antioxidant, an inhibitor of glycosylation, and an endothelin B receptor inhibitor.

In another aspect, the invention relates to a pharmaceutical composition comprising a combination of an AA₁RA (e.g. KW-3902), and an anticonvulsant, as described above, and a physiologically acceptable carrier, diluent, or excipient, or a combination thereof.

Pharmaceutical Compositions

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternatively, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g. by means of conventional mixing, dissolving, granulating, dragee-making, levigating, emulsifying, encapsulating, entrapping or tabeleting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g. in Remington's Pharmaceutical Sciences, above.

For injection, the agents of the invention may be formulated in aqueous solutions or lipid emulsions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Furthermore, the formulations of the present invention may be coated with enteric polymers. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g. dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g. gelatin for use in an inhaler or insulator may be formulated containing a powder mix of the compound and a suitable powder base such as lactose or starch.

The compounds may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g. sterile pyrogen-free water, before use.

The compounds may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant POLYSORBATE 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some emulsions used in solubilizing and delivering the xanthine derivatives described above are discussed in U.S. Pat. No. 6,210,687, and U.S. Patent Application No. 60/674,080, the disclosures of which are each hereby incorporated by reference in their entirety, including any drawings.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g. Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient.

The daily dosage regimen for an adult human patient may be, for example, an oral dose of between 0.1 mg and 500 mg, preferably between 1 mg and 250 mg, e.g. 5 to 200 mg or an intravenous, subcutaneous, or intramuscular dose of between 0.01 mg and 100 mg, preferably between 0.1 mg and 60 mg, e.g. 1 to 40 mg of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

### Example 1

### Administration of KW-3902 and an anticonvulsant in Subjects with Acute CHF and Renal Impairment

More than 275 subjects hospitalized due to acute CHF requiring intravenous diuretic therapy to treat fluid overload, and presenting with creatinine clearance values between 20 to 80 mL/min were identified. Subjects presenting a history of stroke within two years, brain surgery within two years, closed head injury with loss of consciousness over 30 minutes, encephalitis/meningitis within two years, serum sodium levels greater than 128 mmol/L history of seizure, brain tumor, history of penetrating head trauma, history of alcohol withdrawal seizure, advanced Alzheimer's disease, or advanced multiple sclerosis. were classified as “intermediate seizure risk.” Approximately 50 subjects were identified as having an “intermediate seizure risk.” The subjects were randomized to receive either placebo, or 10 mg, 20 mg or 30 mg intravenous KW-3902 per day. The subjects' BNP levels, NT-Pro-BNP, serum creatinine, weight, and NYHA Class were measured. Subjects with an intermediate seizure risk were administered Lorazepam within 72 hours prior to the first treatment with KW-3902 or placebo, or on any treatment day.

Overall, individuals treated with KW-3902 Subjects were categorized as “Failure,” if after Day 7, the subject died, was readmitted to the hospital for heart failure, exhibited worsening heart failure requiring rescue therapy, or exhibited an increase in serum creatinine levels grater than or equal to 0.3 mg/dL at the earlier of hospital discharge or Day 7, compared to the subject's baseline creatinine levels (Day 1).

Subjects were categorized as “Success” if the subject was not characterized as Failure, and if on either day 2 or 3, the subject reported markedly or moderately improved dyspnea, and the investigator reported that the subject had improved such that intravenous diuretic therapy could be converted to oral diuretic therapy.

Subjects were categorized as “Unchanged” if the subject was not categorized as a “Success” or “Failure.”

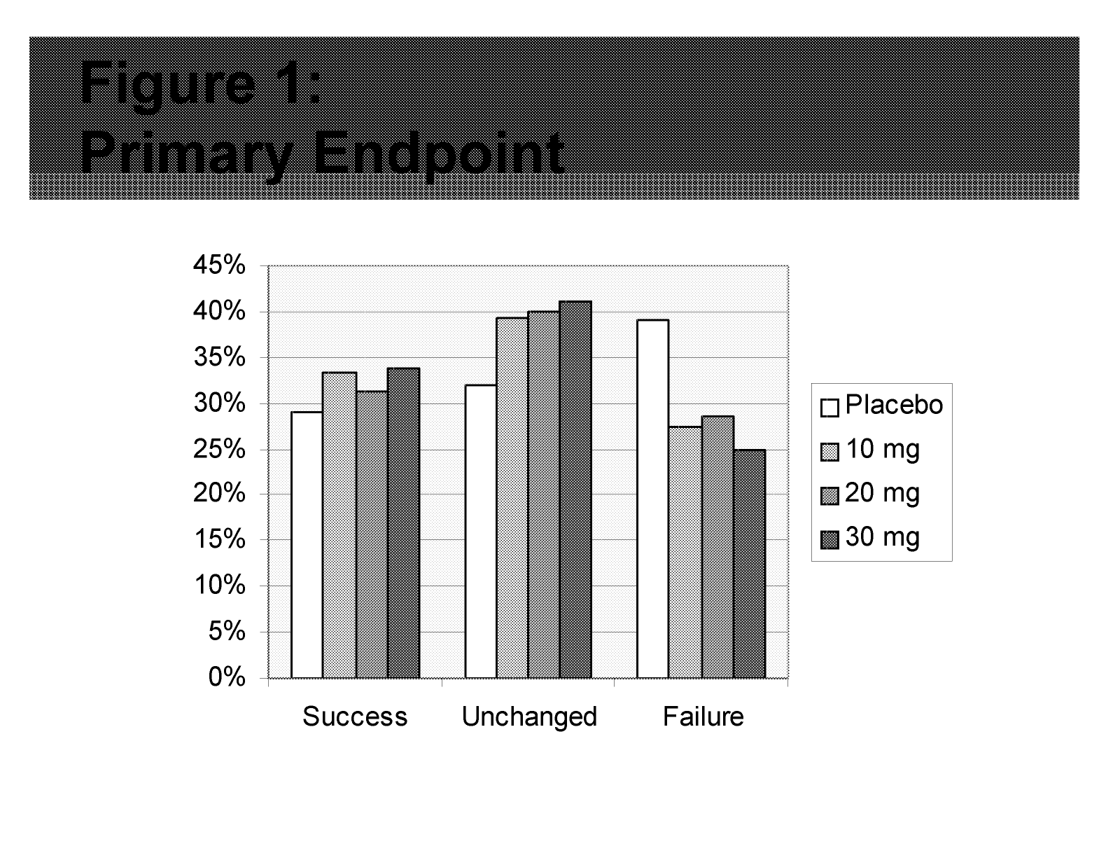

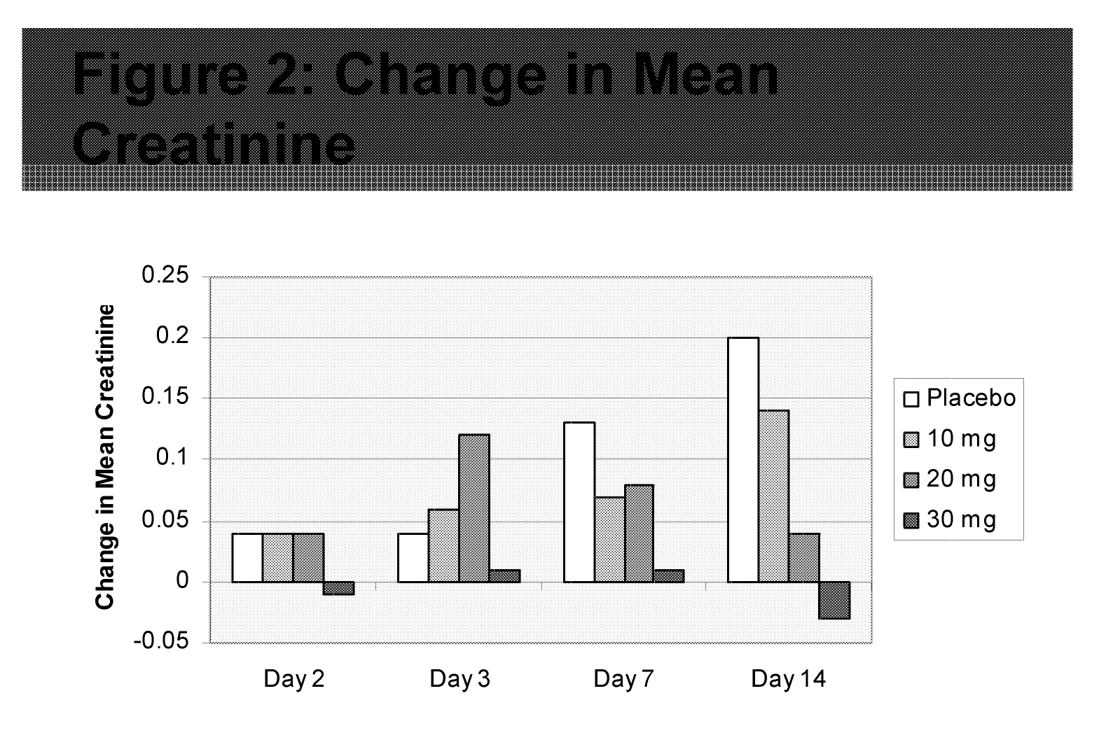

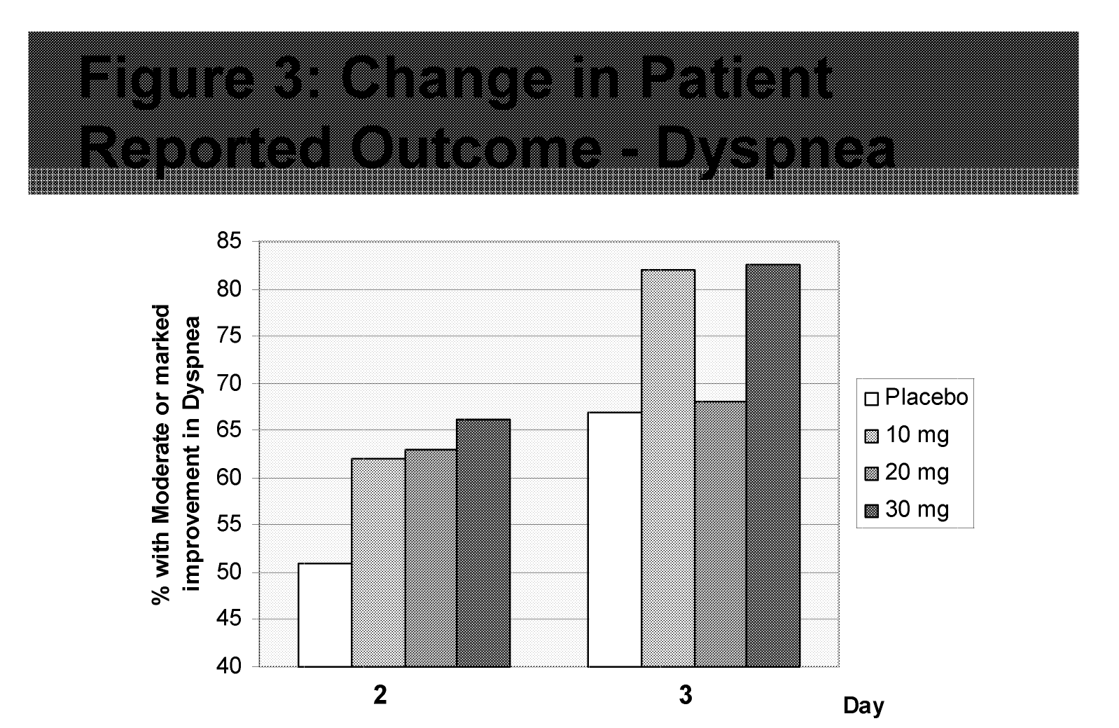

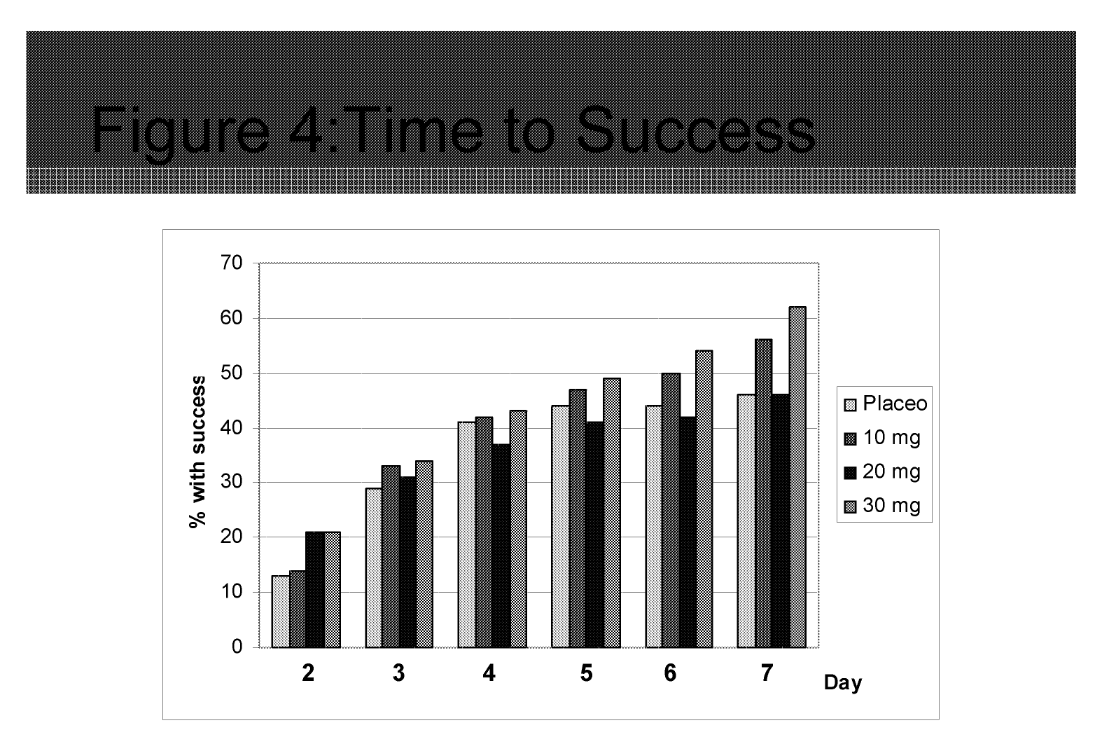

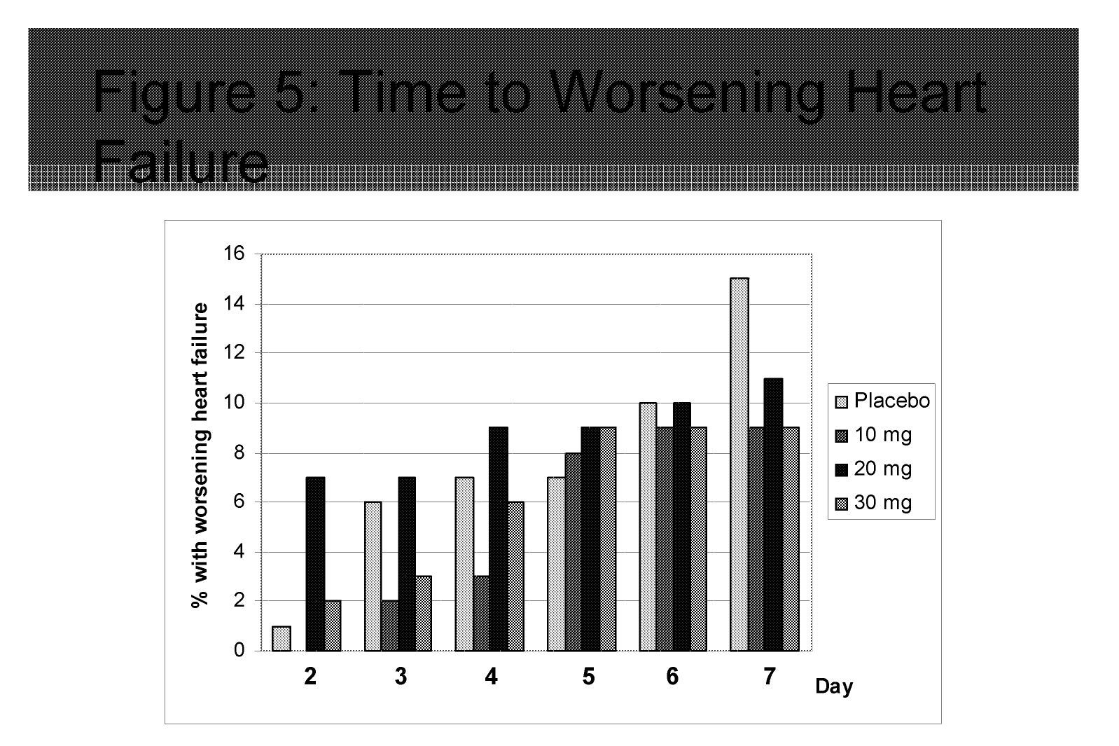

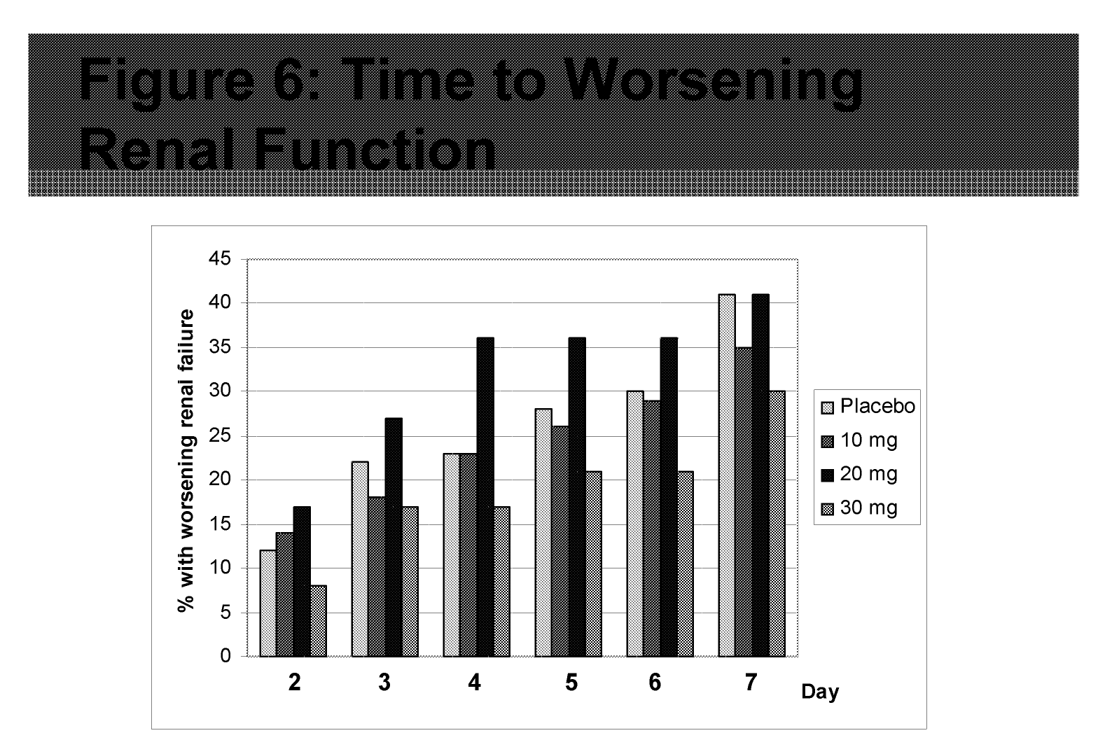

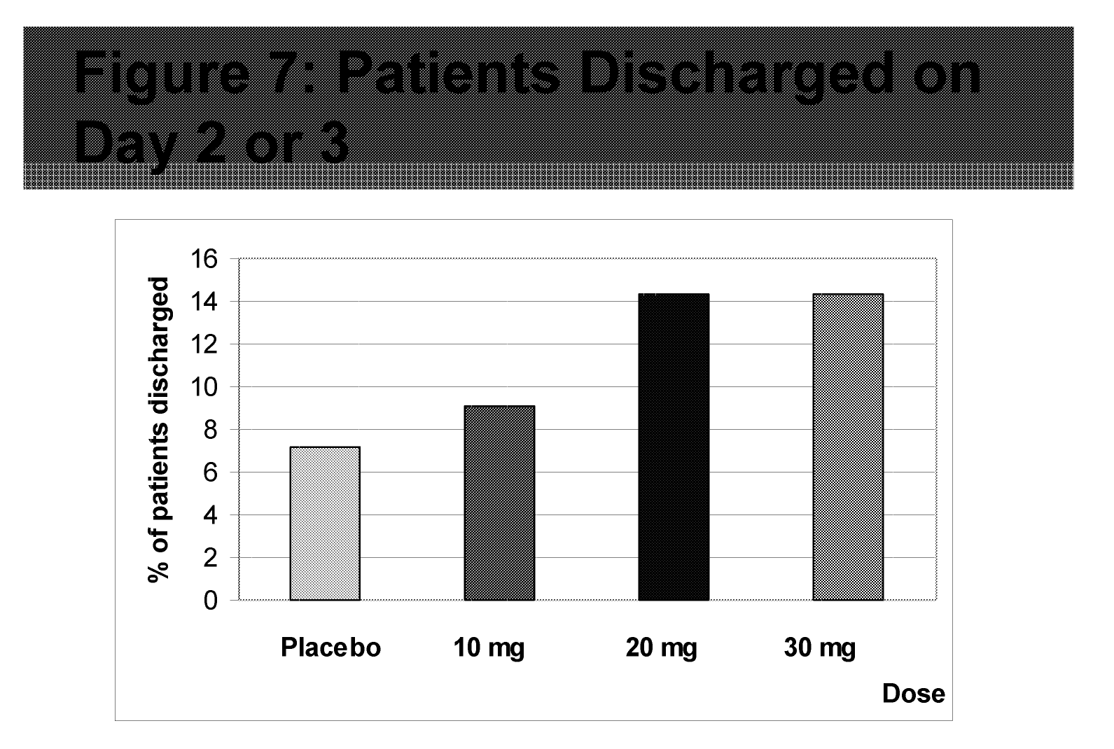

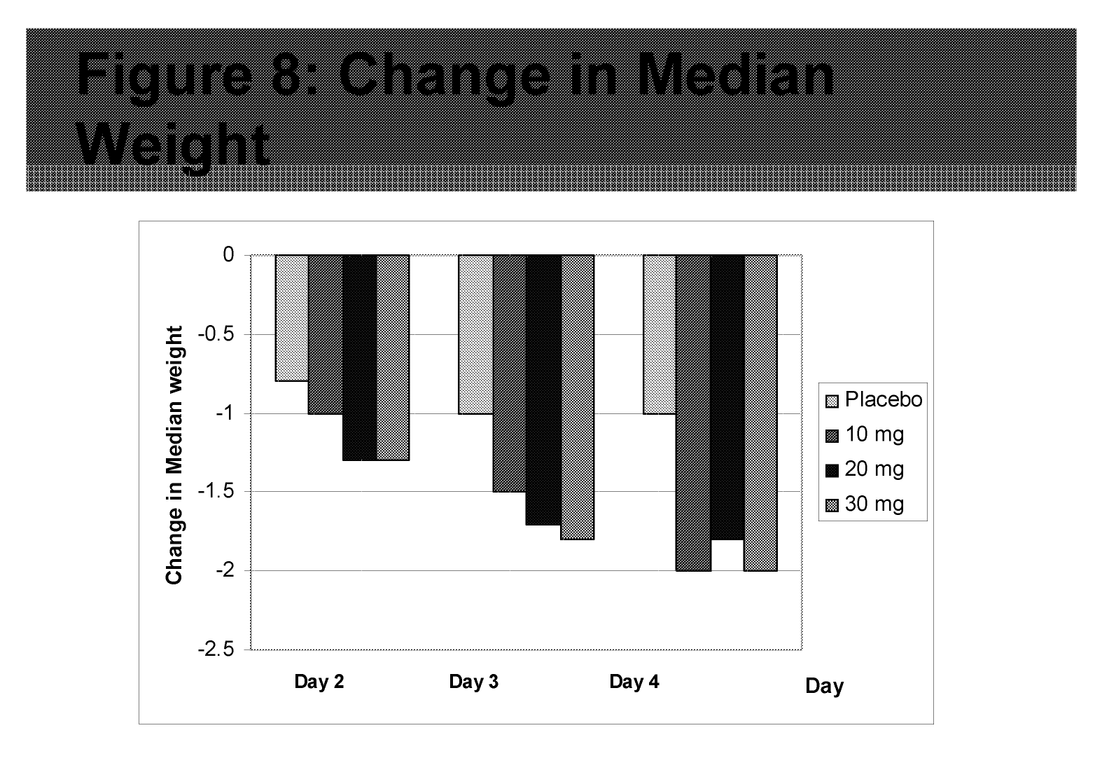

The data from the study are presented in FIGS. 1-8. The percentage of subjects classified as “Failure” was reduced in all three KW-3902 treatment groups as compared to subjects receiving placebo (FIG. 1). Subjects receiving placebo exhibited a greater increase in serum creatinine levels over time. Notably, subjects that received 30 mg KW-3902 showed an overall decrease in serum creatinine levels, indicative of an improvement in renal function at Day 14, whereas subjects that received placebo showed a mean increase in serum creatinine levels at Day 14, indicative of worsening renal function (FIG. 2). Notably, though KW-3902 was only administered over Days 1, 2, and 3, the improvement in serum creatinine levels was observed on Day 14, demonstrating that KW-3902 has a persistent effect on renal function. A greater percentage of subjects reported moderate or marked improvement in dyspnea in groups that received KW-3902 therapy versus in groups that received placebo (FIG. 3). FIG. 4 shows the percentage of subjects characterized as “Success” as described above over time. By Day 7, a higher percentage of the group of subjects treated with 30 mg KW-3902 that were ultimately characterized as “Success” were so characterized at Day 7, compared to the percentage of “Success” subjects that were treated with placebo. In other words, subjects treated with 30 mg KW-3902 improved more quickly than subjects treated with placebo. FIG. 5 shows the percentage of subjects identified as having worsening heart failure in each treatment group over time. By Day 7 a higher percentage of subjects were identified as having worsening heart failure in the placebo treatment group compared to the groups that were treated with KW-3902. FIG. 6 shows the percentage of subjects identified as having worsening renal function in each treatment group over time. On days 2 through 7, a higher percentage of subjects were identified as having worsening renal function in the placebo treatment group compared to the group that was treated with 30 mg KW-3902. FIG. 7 shows that a greater percentage of patients were discharged on days 2 or 3 in subjects that were treated with KW-3902 compared to subjects that were treated with placebo. FIG. 8 shows that subjects who received treatment with KW-3902 showed a greater mean weight loss in on days 2, 3, and 4 compared to subjects treated with placebo.

### Example 3

### Treatment of Individuals with Fluid Overload and Renal Impairment

A subject with fluid overload, as manifested by peripheral edema, dyspnea, and/or other signs or symptoms presents to the hospital, clinic, or doctor's office. The subject also shows some degree of renal impairment and is identified as being at risk for seizure or convulsions upon administration of KW-3902. “At risk” subjects are classified based on history of any one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

In addition to standard of care therapy which would include IV diuretics, e.g. IV furosemide, bumetamide and/or oral metolazone, the subject is also given an amount of KW-3902 between 2.5 mg and 60 mg in injectable form and an anticonvulsant (e.g., a benzodiazepine such as Lorazepam). The subject is administered the dose of KW-3902 and 40 mg of furosemide at 24 hour intervals or more frequently as needed. The subject's fluid intake and output, urine volume, serum and urine creatinine levels, electrolytes and cardiac function are monitored.

At the discretion of the attending physician, the dosage of KW-3902 can be increased or decreased during the treatment. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 4

### Treatment of Individuals Refractory to Standard IV Diuretic Therapy

Subjects presenting with congestive heart failure who were refractory to high dose diuretic therapy, who have an estimated creatinine clearance between 20 mL/min and 80 mL/min are identified. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis. Identified subjects are administered between 2.5 and 100 mg KW-3902, and preferably about 30 mg KW-3902 IV, as well as an anticonvulsant (e.g. a benzodiazepine such as Lorazepam). Changes in urine output and creatinine clearance rate are measured.

A hospitalized subject who has been treated with maximum amounts of IV diuretic and is still symptomatic, fluid overloaded, or whose urine output is less than fluid intake is evaluated for further treatment. A dose of KW-3902 between about 2.5 mg and 100 mg, preferably about 30 mg, in injectable form is infused through the IV line. The subject receives continued treatment with furosemide, and also receives doses of KW-3902 at 6 hour intervals, or more or less frequently as needed. The subject receives continued treatment with anticonvulsants as needed. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 5

### Treatment of Individuals Refractory to Standard IV Diuretic Therapy

A hospitalized subject who has been treated with maximum amounts of IV diuretic and is still symptomatic, fluid overloaded, or whose urine output is less than fluid intake is evaluated for further treatment. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

A dose of about 2.5 mg to about 100 mg KW-3902, preferably about 30 mg KW-3902 in injectable form is infused through the IV line and an anticonvulsant. The subject receives continued treatment with furosemide, and also receives doses of KW-3902 at 6 hour intervals, or more or less frequently as needed. The subject also receives anticonvulsants as needed. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 6

### Treatment of Individuals with Fluid Overload

A subject with fluid overload, as manifested by peripheral edema, dyspnea, and/or other signs or symptoms presents to the hospital, clinic, or doctor's office. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

In addition to standard of care therapy which would include IV diuretics, e.g. IV furosemide, bumetamide and/or oral metolazone, the subject is also given a dose of about 2.5 mg to about 100 mg, preferably about 30 mg of KW-3902 in injectable form and an anticonvulsant. The subject is administered doses of KW-3902 and 40 mg of furosemide at 24 hour intervals, or furosemide can be given as a continuous infusion. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 7

### Treatment of Individuals with Fluid Overload and Impaired Renal Function

A subject with fluid overload, as manifested by peripheral edema, dyspnea, and/or other signs or symptoms presents himself to the physician's office or clinic. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

The subject has been on a therapy regimen that includes oral diuretics and, in addition, to needing a higher dose of diuretics to manage his/her fluid balance, the subject is now showing impaired renal function. The subject is prescribed 5 mg of KW-3902 to be taken orally, once daily, concurrent with other diuretic therapy, as well as an anticonvulsant. The subject's fluid intake and output, urine volume, serum and urine creatinine levels, electrolytes and cardiac function are monitored.

At the discretion of the attending physician, the dosage of KW-3902 can be increased or decreased during the treatment as needed. The dosage of anticonvulsants can be adjusted as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 8

### Treatment of Individuals with Fluid Overload

A subject with fluid overload, as manifested by peripheral edema, dyspnea, and/or other signs or symptoms presents to the physician's office or clinic. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

The subject has been on a therapy regimen that includes oral diuretics and needs a higher dose of diuretics to manage his/her fluid balance. To delay or prevent the onset of renal impairment and/or to delay the need to use higher dosages of standard diuretics, the subject is prescribed about 2.5 to about 100 mg of KW-3902, preferably about 30 mg KW-3902 to be taken orally, once daily, concurrent with their diuretic therapy. The subject is also provided an anticonvulsant (e.g., a benzodiazepine such as Lorazepam). The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 9

### Treatment of Individuals with Congestive Heart Failure

A subject with congestive heart failure presents to the physician's office or clinic. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

The subject is put on a therapy regimen that includes oral diuretics to manage his/her fluid balance. To delay or prevent the onset of renal impairment and/or to delay the need to use higher dosages of standard diuretics, the subject is also prescribed 5 mg of KW-3902 to be taken orally, once daily, concurrent with their diuretic therapy. The subject is also provided an anticonvulsant, (e.g., a benzodiazepine such as Lorazepam). The subject's fluid levels, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 10

### Improving Health Outcomes for of Individuals with Congestive Heart Failure

A subject with congestive heart failure presents to the physician's office or clinic. The subject is identified as being “at risk” for developing a seizure or convulsions. Individuals characterized as being “at risk” for developing seizure or convulsions include individuals presenting with at least one of the following: history of seizure, stroke within two years, brain tumor, brain surgery within two years, encephalitis within two years, history of penetrating head trauma, closed head injury with loss of consciousness over 30 minutes within two years, history of alcohol withdrawal seizure, advanced Alzheimer's disease, or advanced multiple sclerosis.

The subject is put on a therapy regimen that includes oral diuretics to manage his/her fluid balance. To improve overall health outcomes (i.e., morbidity or mortality rates due to CHF), the subject is also prescribed 5 mg of KW-3902 to be taken orally, once daily, concurrent with their diuretic therapy, or similar doses of KW-3902 is administered to the subject intravenously. The subject is also provided an anticonvulsant (e.g. a benzodiazepine such as Lorazepam). The subject's fluid levels, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

What is claimed is:

1. A pharmaceutical composition comprising an effective prophylactic amount of an anticonvulsant and a therapeutically effective amount of an adenosine A₁ receptor antagonist (AA₁RA).

2. The composition of claim 1, wherein said anticonvulsant is selected from the group consisting of diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof.

3. The composition of claim 1, wherein said anticonvulsant is diazepam or lorazepam.

4. The composition of claim 1, further comprising a non adenosine-modifying diuretic.

5. The composition of claim 1, wherein the AA₁RA is KW-3902.

6. The composition of claim 1, wherein said AA₁RA is a xanthine-derivative compound of Formula I or a pharmaceutically acceptable salt thereof, 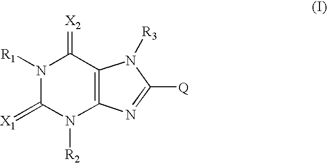
wherein
each of X₁ and X₂ independently represents oxygen or sulfur;
Q represents: 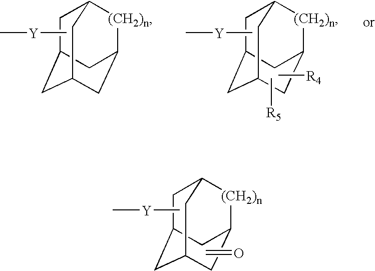
where Y represents a single bond or alkylene having 1 to 4 carbon atoms, n represents 0 or 1;
each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or
R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 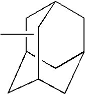
then R₁, R₂ and R₃ are not simultaneously methyl.

7. The composition of claim 6, wherein both of R₁ and R₂ are lower alkyl and R₃ is hydrogen; and both of X₁ and X₂ are oxygen.

8. The composition of claim 6, wherein each of R₁, R₂ and R₃ independently represents hydrogen or lower alkyl.

9. The composition of claim 6, wherein each of R₁ and R₂ independently represents allyl or propargyl and R₃ represents hydrogen or lower alkyl.

10. The composition of claim 6, wherein R₁ is hydroxy-substituted, oxo-substituted or unsubstituted propyl; R₂ is hydroxy-substituted or unsubstituted propyl; and Y is a single bond.

11. The composition of claim 6, wherein R₁ is propyl, 2-hydroxypropyl, 2-oxopropyl or 3-oxopropyl; R₂ is propyl, 2-hydroxypropyl or 3-hydroxypropyl.

12. The composition of claim 9, wherein X₁ and X₂ are both oxygen and n is 0.

13. The composition of claim 8, wherein Q is

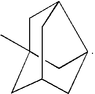

14. The composition of claim 8, wherein Q is

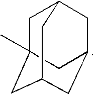

15. The composition of claim 6, wherein Q is 9-hydroxy, 9-oxo or 6-hydroxy substituted 3-tricyclo[3.3.1.0^{3,7}]nonyl, or 3-hydroxy-1tricyclo[3.3.1.1^{3,7}]decyl.

16. The composition of claim 6, wherein said AA₁RA is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine, 8-(cis-9-hydroxy-3-tricyclo[3.3.1.0^{3,7}]nonyl)-1,3-dipropylxanthine, 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0³⁷]nonyl)-3-propylxanthine, or a pharmaceutically acceptable salt thereof.

17. The composition of claim 6, wherein said AA₁RA is a xanthine epoxide-derivative compound of Formula II or Formula III, or a pharmaceutically acceptable salt thereof, 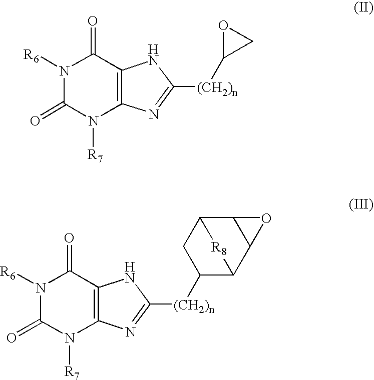
wherein R₆ and R₇ are the same or different, and can be hydrogen or an alkyl group of 1-4 carbons, R₈ is either oxygen or (CH₂)₁₋₄, and n=0-4.

18. The composition of claim 17, wherein said xanthine epoxide-derivative compound is

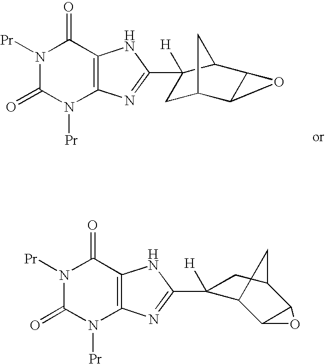

19. The composition of claim 1, further comprising a beta-blocker.

20. The composition of claim 1, further comprising an angiogtensin converting enzyme (ACE) inhibitor.

21. The composition of claim 1, further comprising and angiogtensin II receptor blocker (ARB).

22. The composition of claim 21, further comprising an ACE inhibitor.

23. A method of providing AA₁RA therapy to a patient, comprising:
identifying a patient in need thereof, and
administering to said patient a therapeutically effective amount an AA₁RA, or a pharmaceutically acceptable salt, ester, amide, metabolite, or prodrug thereof, in conjunction with an effective prophylactic amount of an anticonvulsant agent.

24. The method of claim 23, further comprising administering a non adenosine-modifying diuretic to said individual.

25. The method of claim 23, wherein said anticonvulsant is selected from the group consisting of diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof.

26. The method of claim 23, wherein said anticonvulsant is diazepam or lorazepam.

27. The method of claim 24, wherein the non adenosine-modifying diuretic is selected from the group consisting of hydrochlorothiazides, furosemide, torsemide, bumetamide, ethacrynic acid, piretamide, spironolactone, triamterene, and amiloridethiazides.

28. The method of claim 23, wherein the AA₁RA is KW-3902.

29. The method of claim 23, wherein said AA₁RA is a xanthine-derivative compound of Formula I or a pharmaceutically acceptable salt thereof, 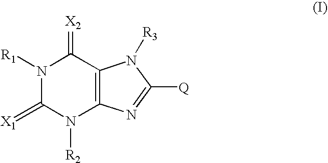
wherein
each of X₁ and X₂ independently represents oxygen or sulfur;
Q represents: 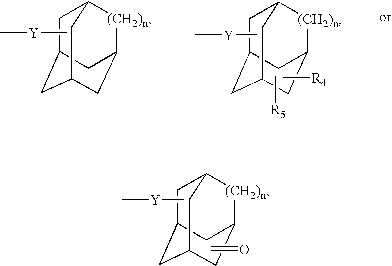
where Y represents a single bond or alkylene having 1 to 4 carbon atoms, n represents 0 or 1;
each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or
R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 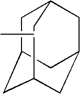
then R₁, R₂ and R₃ are not simultaneously methyl.

30. The method of claim 29, wherein both of R₁ and R₂ are lower alkyl and R₃ is hydrogen; and both of X₁ and X₂ are oxygen.

31. The method of claim 29, wherein each of R₁, R₂ and R₃ independently represents hydrogen or lower alkyl.

32. The method of claim 29, wherein each of R₁ and R₂ independently represents allyl or propargyl and R₃ represents hydrogen or lower alkyl.

33. The method of claim 29, wherein R₁ is hydroxy-substituted, oxo-substituted or unsubstituted propyl; R₂ is hydroxy-substituted or unsubstituted propyl; and Y is a single bond.

34. The method of claim 29, wherein R₁ is propyl, 2-hydroxypropyl, 2-oxopropyl or 3-oxopropyl; R₂ is propyl, 2-hydroxypropyl or 3-hydroxypropyl.

35. The method of claim 32, wherein X₁ and X₂ are both oxygen and n is 0.

36. The method of claim 31, wherein Q is

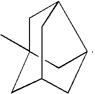

37. The method of claim 31, wherein Q is

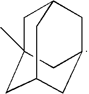

38. The method of claim 29, wherein Q is 9-hydroxy, 9-oxo or 6-hydroxy substituted 3-tricyclo[3.3.1.0^{3,7}]nonyl, or 3-hydroxy-1-tricyclo[3.3.1.1^{3,7}]decyl.

39. The method of claim 29, wherein said AA₁RA is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine, 8-(cis-9-hydroxy-3-tricyclo[3.3.1.0^{3,7}]nonyl)-1,3-dipropylxanthine, 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine, or a pharmaceutically acceptable salt thereof.

40. The method of claim 29, wherein said AA₁RA is a xanthine epoxide-derivative compound of Formula II or Formula III, or a pharmaceutically acceptable salt thereof, 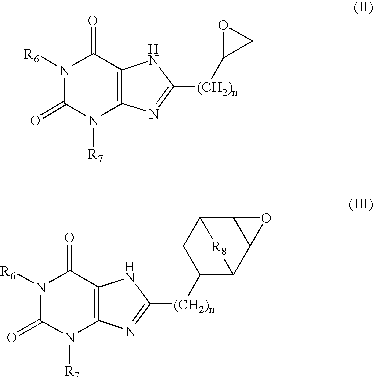
wherein R₆ and R₇ are the same or different, and can be hydrogen or an alkyl group of 1-4 carbons, R₈ is either oxygen or (CH₂)₁₋₄, and n=0-4.

41. The method of claim 40, wherein said xanthine epoxide-derivative compound is

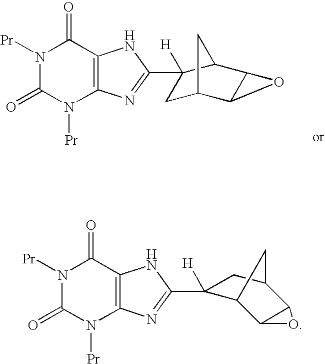

42. In a method of administering an AA₁RA to a subject in need thereof, the improvement comprising also administering to said subject an effective prophylactic amount of an anticonvulsant.
